(19) United States
(12) Patent Application Publication
(10) Pub. No.: US 2024/0225518 A1
(43) Pub. Date: Jul. 11, 2024

(54) MEDICAL SYSTEM AND METHOD FOR MEASURING VISUAL EVOKED POTENTIALS (VEP) OF A PERSON

(71) Applicant: UNIVERSITÄT ZÜRICH, Zürich (CH)

(72) Inventors: Valentine MARCAR, Zürich (CH); Marcus CHEETHAM, Birmensdorf (CH)

(73) Assignee: UNIVERSITÄT ZÜRICH, Zürich (CH)

(21) Appl. No.: 18/558,745

(22) PCT Filed: May 3, 2023
(86) PCT No.: PCT/EP2022/061870

§ 371 Date: Nov. 3, 2023

(30) Foreign Application Priority Data
May 3, 2021 (EP) 21171898.6

(51) Int. Cl. A61B 5/378; A61B 3/113; A61B 5/00; A61B 5/291; A61B 5/372

(52) U.S. Cl. CPC A61B 5/378; A61B 3/113; A61B 5/291; A61B 5/372; A61B 5/6814; A61B 5/7425

(57) ABSTRACT

The present invention relates to a medical system (20) for measuring visual evoked potentials (VEP) of a person, comprising a display (2) for displaying images to be observed by the person with a single eye, and an image generating module (1), the image generating module (1) being configured to cause the display (2) to display a sequence of images comprising a first image and a second image. The medical system (20) further comprises: a plurality of electrodes (3) configured to be attached to the scalp of the person and to detect EEG signals indicative of electrical activity in the brain of the person in response to observing said sequence of images displayed by the display (2), an EEG recording module (4) connected to said plurality of electrodes (3), the EEG recording module (4) being configured to record said EEG signals, and an analyzing module (8), the analyzing module (8) being configured to derive from said EEG signals recorded by the EEG recording module (4) at least one visual evoked potential (VEP).

The present invention relates to a system and a method for measuring visual evoked potentials (VEP) of a brain of a person.

The amplitude of the early VEP components directly reflects the size of the neural population that is active during processing of the temporal luminance contrast.

Particularly, the amplitude of individual VEP deflections, allows determining the functionality of the macula of the retina in case the stimulus requires a resolution that can only be achieved in the macula. Thus, diseases relating to the macula can have an effect on the VEP. On the other hand, also damages of the optic nerve and complete visual pathway can alter the VEP.

Therefore, it is an objective of the present invention to provide a medical system that allows to measure VEP of a person, that provides an improved means enabling a more precise determination of the state of health of the retina and an analysis thereof, i.e., for diagnostic purposes.

This problem is solved by a medical system having the features of claim 1 and a method having the features of claim 20.

Preferred embodiments of these aspects of the present invention are stated in the corresponding sub claims and are described below.

According to claim 1, a medical system for measuring visual evoked potentials (VEP) of a person is disclosed, comprising:
a display for displaying images to be observed by the person with a single eye,
an image generating module, the image generating module being configured to cause the display to display a sequence of images comprising a first image and a second image, the first and the second image being displayed alternately, wherein the first image comprises a windmill pattern consisting of a number N of equidistantly spaced light circular sectors, and a number of N equidistantly spaced dark circular sectors, wherein the number N is selected from the set comprised of 4, 8, 16, 32, and wherein the light and the dark circular sectors each extend outwards in a radial direction from a common central point, wherein each dark circular sector is arranged between two light circular sectors, wherein the second image corresponds to the first image, but with the order of the light circular sectors and the dark circular sectors (32) being reversed, or wherein the second image corresponds to an all-dark area having the same color and luminance as the dark circular sectors,
a plurality of electrodes configured to be attached to the scalp of the person and to detect EEG signals indicative of electrical activity in the brain of the person in response to observing said sequence of images displayed by the display,
an EEG recording module connected to said plurality of electrodes, the EEG recording module being configured to record said EEG signals, and
an analyzing module, the analyzing module being configured to derive from said EEG signals recorded by the EEG recording module at least one visual evoked potential (VEP).

Particularly, the display can be a computer monitor, the image generating module can be formed by a computer on which a computer program is executed causing the display to display said sequence of images. Furthermore, the analyzing module can be formed by the computer or by a further computer on which a computer program is executed that derives the VEP from the recorded EEG signals. Particularly, the EEG recording module to which the electrodes are connected can be connected to the computer representing the analyzing module via an interface of the computer. The EEG recording module can convert the analog EEG signals into digital signals that can be input into the computer via said interface and processed by the computer program associated with the analyzing module.

Alternatively, the image generating module and the analyzing module can each also be formed by a dedicated hardware, respectively.

With the measurement of visual evoked potentials (VEP), it is possible to study the function of the visual pathway from the eye to the visual cortex.

Particularly, the VEP is a derivative of the EEG of the person obtained by averaging the EEG signals time-locked to the presentation of a visual stimulus, e.g., an on- or offset of said first image in said sequence of images. The averaging of EEG signals allows to suppress interference and enhances the stimulus responses by the visual cortex. Preferably at least two complementary images are used during examination. For each image the VEP is obtained in form of a stimulus curve comprising characteristic deflections, the most important deflection being a positive wave occurring about 100 ms after presentation of the stimulus, which is denoted as P100. Particularly, in diseases, the time of occurrence (latency) of the P100 component can be delayed, its amplitude reduced, or the P100 component may be absent altogether.

Since the information from both eyes of the person arrive in the same visual cortex, both eyes have to be measured separately from one another when deriving the VEP from the EEG data.

According to a preferred embodiment of the medical system the light circular sectors are white circular sectors and the dark circular sectors are preferably black circular sectors.

Furthermore, according to an embodiment of the medical system, the light circular sectors comprise a luminance of e.g. 167 cd/m². Furthermore, the dark circular sectors comprise a luminance of e.g. 0.015 cd/m² and/or wherein the Michelson contrast between light and dark circular sectors (31, 32) is at least 40.

Furthermore, according to an embodiment, the Michelson contrast between light and dark circular sectors preferably is in the range from 33 to 99. According to an embodiment, the Michelson contrast is 99.8.

According to a further embodiment of the medical system according to the present invention, each light circular sector is delimited by two radii extending from the common central point in the radial direction and an arc extending in a circumferential direction. Preferably, the arcs of the light circular sectors are of equal length.

According to yet another preferred embodiment of the medical system, the light and dark circular sectors together define a circle, particularly with the common central point forming the center of the circle.

Preferably, according to an embodiment, the first image comprises a region adjacent the circle, said region having the same color and luminance as the dark circular sectors so that the dark circular sectors seamlessly blend into said region of the first image. Particularly, this means that the arcs of the dark circular sectors cannot be perceived, but the circle is still well-defined by virtual continuation of the arcs of the light circular sectors across the dark circular sectors.

Furthermore, according to an embodiment of the medical system, the light circular sectors make up a percentage of the total area of the circle, wherein this percentage is one of: 12.5%, 25%, 37.5%, 50%, 75%. According to a preferred embodiment, said percentage is 50%. According to a most preferred embodiment, the windmill pattern comprises 4 light circular sectors. Smaller differences between the percentages than 12.5% are also conceivable.

According to a preferred embodiment of the medical system, the image generating module is configured to cause the display to display a series of sequences for different percentages of the area of the circle. Particularly, the different percentages are: 12.5%, 25%, 37.5%, 50%, and 75%.

Preferably, according to an embodiment, each first image and each second image in the sequence is displayed over a constant period of time, starting with an onset of the respective image and ending with an offset of the respective image.

Preferably, in an embodiment, said constant period of time from onset to offset of the respective first or second image is at least 400 ms, preferably 500 ms.

According to the medical system according to the present invention, the second image corresponds to the first image, but with the order of the light circular sectors and the dark circular sectors being reversed. Such a sequence of images, where the first and the second image are shown in an alternating fashion is also denoted as pattern alternating display.

Particularly, showing the first and second image in an alternating fashion means that at those positions where formerly a light circular sector started and extended in the clockwise circumferential direction of the circle, now a dark sector starts and extends in the clockwise circumferential direction. Thus, the sequence of alternating first and second images generates the impression that the light and dark circular sectors are instantly rotated back and forth, i.e., clockwise and counter-clockwise, in an alternating fashion.

Particularly, regarding the pattern alternating display, the analyzing module is configured to determine a visual evoked potential (VEP) by averaging a pre-defined number of EEG signal segments following onset of the first and/or second images in the sequence, each segment preferably having a duration corresponding to said constant period of time, e.g. 500 ms.

According to the medical system of this invention, the sequence of images may alternatively correspond to a sequence, in which only the first image is shown repeatedly, wherein a second image is shown between each to successive first images, the second image corresponding to an all-dark area, particularly making up the complete display area, wherein said dark area comprises the same color and luminance as the dark circular sectors. This means, in other words, that all light circular sectors of the first image are replaced by dark circular sectors of the same size as the light circular sectors turning the whole image dark.

Here, the analyzing module is configured to determine a visual evoked potential (VEP) by averaging a pre-defined number of EEG signal segments following onset of the first images in the sequence. Alternatively, or in addition, the analyzing module is configured to determine a visual evoked potential (VEP) by averaging a pre-defined number of EEG signal segments following offset of the first images in the sequence.

Particularly, according to an embodiment, the analyzing module is configured to compare an amplitude of at least one deflection of the considered visual evoked potential (VEP) with an amplitude of a corresponding deflection of a reference curve. Alternatively, or in addition, the analyzing module is configured to compare a latency of at least one deflection of the considered visual evoked potential (VEP) with a latency of a corresponding deflection of the reference curve. Particularly, the amplitude is a maximum of the visual evoked potential within a pre-determined time interval, wherein the latency is the time of the maximum.

According to a preferred embodiment of the medical system according to the present invention, when the sequence of images comprises the second image corresponding to the first image, but with the order of the light circular sectors and the dark circular sectors being reversed, the respective deflection is P100, wherein the amplitude of P100 is defined as the maximum of the visual evoked potential between 70 ms and 140 ms, and wherein the latency of P100 is the time of the maximum.

According to a preferred embodiment of the medical system, when the sequence of images comprises the second image being the all-dark area, the respective deflection is P1, wherein the amplitude of P1 is defined as the maximum of the visual evoked potential between 70 ms and 140 ms, and wherein the latency of P1 is the time of the maximum.

Furthermore, according to a preferred embodiment of the medical system, said reference curve is an average of visual evoked potentials taken from a plurality of persons, each person having an intact retina, the respective visual evoked potential being based on the same sequence of images (see above) presented to the person under examination.

Further, according to a preferred embodiment of the medical system, the analyzing unit is configured to detect that the person has a diseased retina and to provide a corresponding information to an operator of the system, if the amplitude of said at least one deflection of the visual evoked potential deviates from the amplitude of a corresponding deflection of the reference curve by more than a pre-determined amount, and/or if the latency of said at least one deflection of the visual evoked potential deviates from the latency of a corresponding deflection of the reference curve by more than a pre-determined amount.

Furthermore, according to an embodiment of the medical system, the latter preferably comprises a camera configured for tracking movement of the eye of the person being under examination, wherein the image generation module is configured to only display images of said sequence in case the person fixates said common central point with said eye under examination.

According to a preferred embodiment of the medical system, the analyzing unit is configured to detect that the person has a diseased retina based on comparing amplitudes of a P100 deflection of the visual evoked potential (VEP) obtained from displaying a series of sequences for different percentages of the area of the circle to be observed by the person with a reference curve. Particularly, the P100 deflection corresponds to the maximum of the VEP between 70 ms and 140 ms.

According to a further preferred embodiment, the reference curve is a linear function of the percentage of the area of the circle. Preferably, the analyzing unit is configured to detect that the person has a deseased retina if one or several of the amplitudes of the P100 deflections deviate from the reference curve. The reference curve can be obtained from a group of persons having a healthy retina.

Yet another aspect of the present invention relates to a method for measuring visual evoked potentials (VEP) of a person, preferably using a system according to the present invention as described herein, the method comprising the steps of:
a) Causing a display to display a sequence of images to be observed by the person with a single eye, the sequence of images comprising a first image and a second image, the first and the second image being displayed alternately, wherein the first image comprises a windmill pattern consisting of a number N of equidistantly spaced light circular sectors, and a number N of equidistantly spaced dark circular sectors, wherein in an embodiment the number N is preferably selected from the set comprised of 4, 8, 16, 32, and wherein the light and the dark circular sectors each extend outwards in a radial direction from a common central point, wherein each dark circular sector is arranged between two light circular sectors, wherein the second image corresponds to the first image, but with the order of the light circular sectors and the dark circular sectors being reversed, or wherein the second image corresponds to an all-dark area having the same color and luminance as the dark circular sectors,
b) Detecting EEG signals via electrodes attached to the scalp of the person and recording said EEG signals, the EEG signals being indicative of electrical activity in the brain of the person in response to observing said sequence of images, and
c) Deriving at least one visual evoked potential (VEP) of the person from said EEG signals.

According to a preferred embodiment of the method according to the present invention, the light circular sectors are white circular sectors and/or wherein the dark circular sectors are black circular sectors.

Furthermore, according to an embodiment of the method according to the present invention, the light circular sectors comprise a luminance of e.g. 167 cd/m². Furthermore, according to an embodiment, the dark circular sectors comprise a luminance of e.g. 0.015 cd/m² and/or wherein the Michelson contrast between light and dark circular sectors is at least 40 Regarding values of the Michelson contrast see details stated already in conjunction with the apparatus which apply here as well.

As described above in conjunction with the medical system according to the present invention, also in relation to the method according to the present invention, each light circular sector is delimited by two radii extending from the common central point in the radial direction and an arc extending in a circumferential direction. Furthermore, preferably, the arcs of the light circular sectors are of equal length. Further, particularly, the light and dark circular sectors together form a circle. Preferably, according to an embodiment of the method, the first image comprises a region adjacent the circle, said region having the same color and luminance as the dark circular sectors so that the dark circular sectors seamlessly blend into said region of the first image.

Furthermore, according to an embodiment of the method according to the present invention, the light circular sectors make up a percentage of the area of the circle, wherein this percentage is one of: 12.5%, 25%, 37.5%, 50%, 75%. In a preferred embodiment, the display is caused to display a series of sequences for different percentages of the area of the circle (see also above).

According to yet another embodiment of the method according to the present invention, in step a), said sequence of images further comprises a second image, the first and the second image being displayed alternately. Particularly, each first image and each second image in the sequence is displayed over a constant period of time, starting with an onset of the respective image and ending with an offset of the respective image. Preferably, said constant period of time from onset to offset of the respective image is 500 ms.

According to a preferred embodiment of the method, the second image corresponds to the first image, but with the order of the light circular sectors and the dark circular sectors being reversed. In this case, preferably, a visual evoked potential (VEP) is automatically determined according to an embodiment of the method by averaging a pre-defined number of EEG signal segments following onset of the first and/or second images in the sequence, wherein each segment preferably has a duration corresponding to said constant period of time, e.g. 500 ms.

According to an alternative embodiment, the second image corresponds to an all-dark area making up the complete display area and having the same color and luminance as the dark circular sectors (this sequence is also denoted as pattern on/off stimuli). Here, in an embodiment, a visual evoked potential (VEP) is automatically determined by averaging a pre-defined number of EEG signal segments following onset of the first images in the sequence, and/or wherein a visual evoked potential (VEP) is automatically determined by averaging a pre-defined number of EEG signal segments following offset of the first images in the sequence.

Regarding both embodiments, i.e., pattern alternating display and on/off stimuli, an amplitude of at least one deflection of the considered visual evoked potential (VEP) is automatically compared with an amplitude of a corresponding deflection of a reference curve. Alternatively, or in addition, a latency of at least one deflection of the considered visual evoked potential (VEP) is automatically compared with a latency of a corresponding deflection of a reference curve.

According to a preferred embodiment, when the sequence of images comprises the second image corresponding to the first image, but with the order of the light circular sectors and the dark circular sectors being reversed (pattern alternating display), the respective deflection is P100 (see also above).

Furthermore, according to an embodiment, when the sequence of images comprises the second image being the all-dark area (pattern on/off stimuli), the respective deflection is P1 (see also above).

Furthermore, according to an embodiment of the method according to the present invention, a movement of the eye of the person is tracked, particularly by means of a camera, wherein images of said sequence are only displayed in case the person fixates said common central point of the respective windmill pattern with said eye that is to be examined.

According to a preferred embodiment of the method amplitudes of a P100 deflection of the visual evoked potential (VEP) obtained from displaying a series of sequences for different percentages of the area of the circle are compared to a reference curve. According to a preferred embodiment, the reference curve is a linear function of the percentage of the area of the circle (see also above).

Furthermore, yet another aspect of the present invention relates to a computer program, the computer program comprising instructions which, when the program is executed by a computer, cause the computer to carry out step a) of the method according to the present invention.

In the following, embodiments as well as further examples, features and advantages of the present invention will be explained with reference to the Figures, wherein

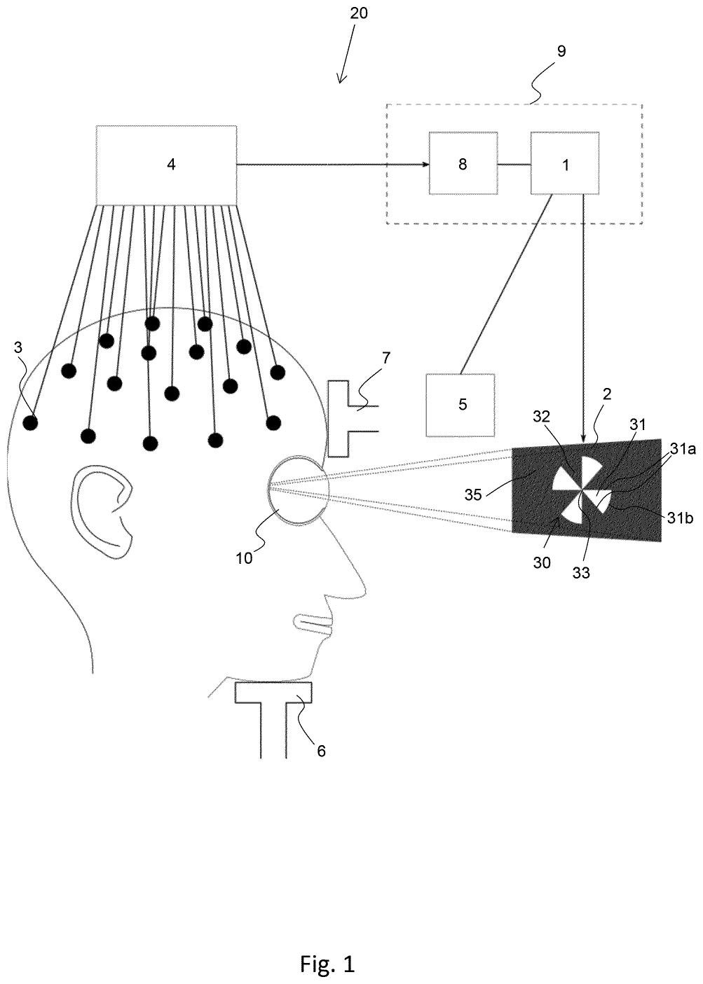

FIG. 1 shows a schematic view of the medical system according to the present invention for measuring visual evoked potentials of a person;

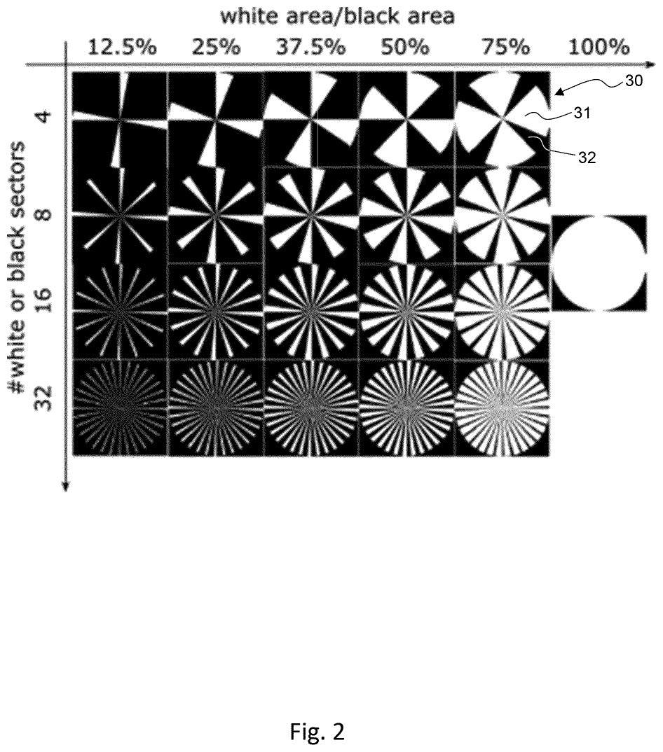

FIG. 2 shows different possible windmill patterns that can used for determining VEP of a person observing the respective pattern, wherein the number of light (e.g. white) and dark (e.g. black) circular sectors can vary between 4, 8, 16, 32, and wherein the light circular sector can assume a fraction of the circle area corresponding to one of 12.5%, 25%, 37.5%, 50%;

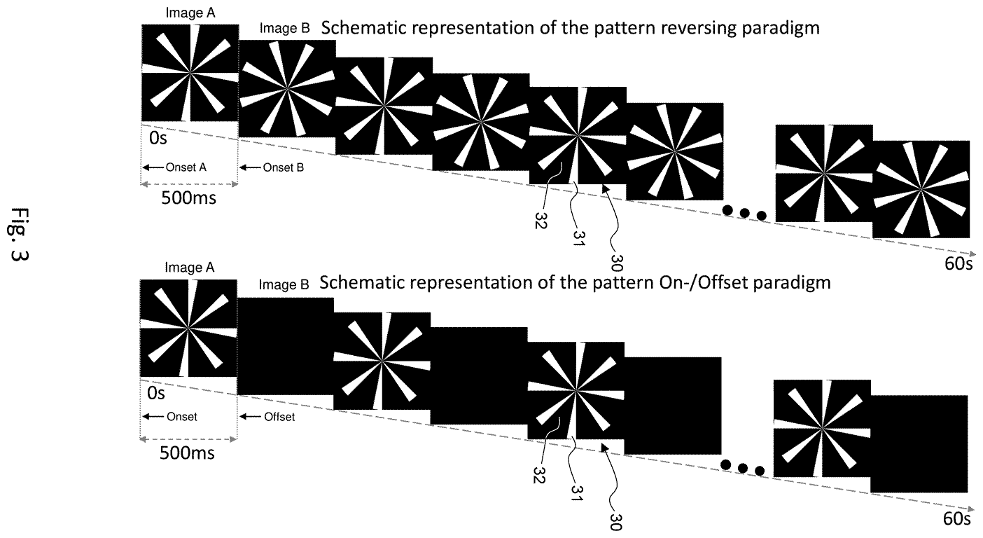

FIG. 3 shows a sequence of a first and a second image in form of windmill pattern, respectively, shown in a pattern alternating display (also denoted as pattern reversing paradigm) as well as pattern on/off stimuli (also denoted as pattern on/off paradigm);

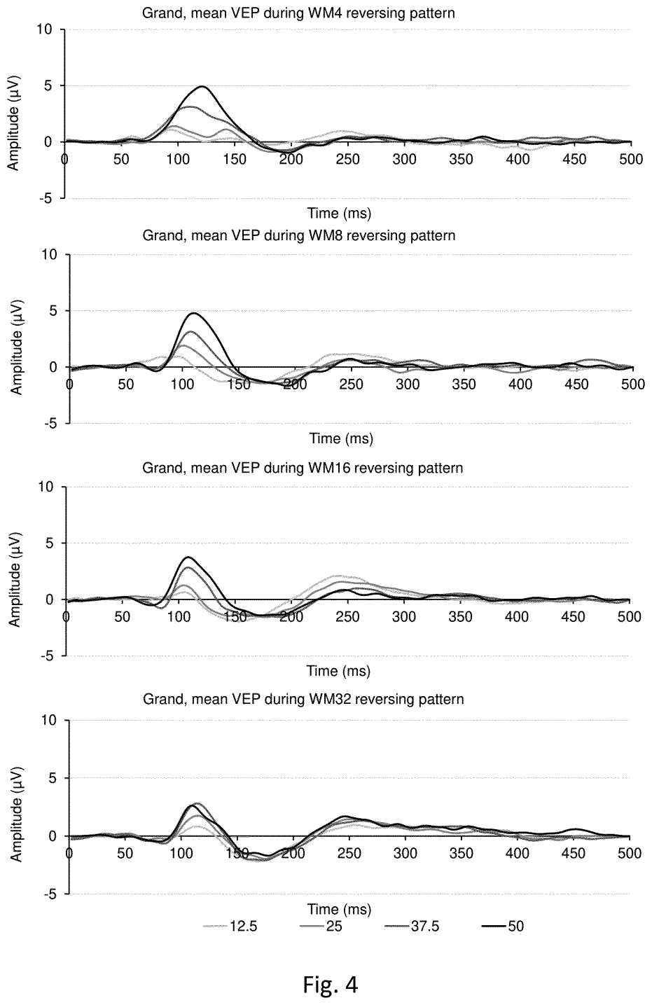

FIG. 4 shows grand mean VEP signals as a function of time, measured using the pattern reversing paradigm for 4, 8, 16 and 32 white or black circular sectors as well as for fractions of 12.5%, 25%, 37.5% and 50% of the circle area being covered by white circular sectors;

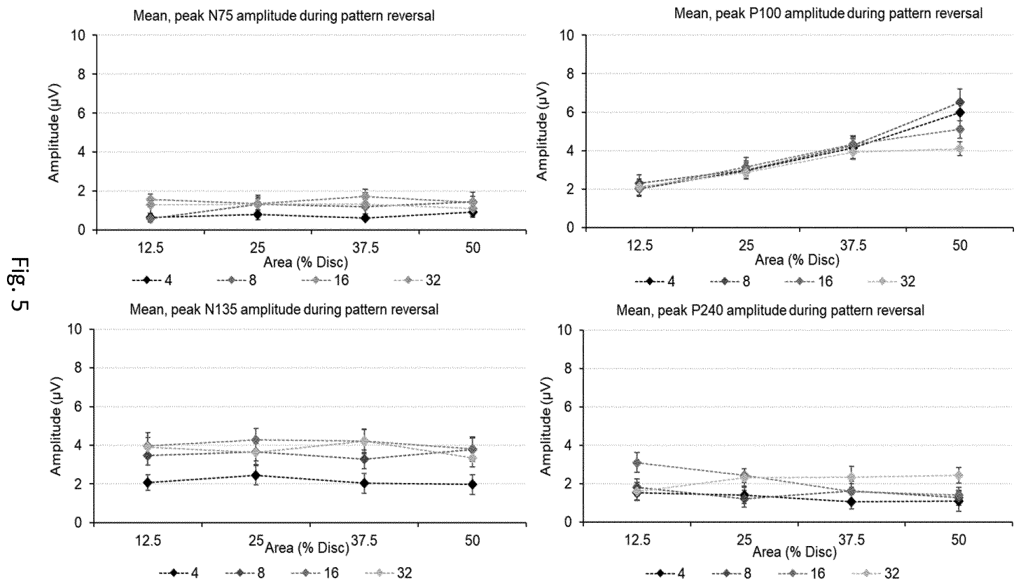

FIG. 5 shows the respective N75, P100, N135 and P240 absolute mean component amplitudes of the VEP signals shown in FIG. 4;

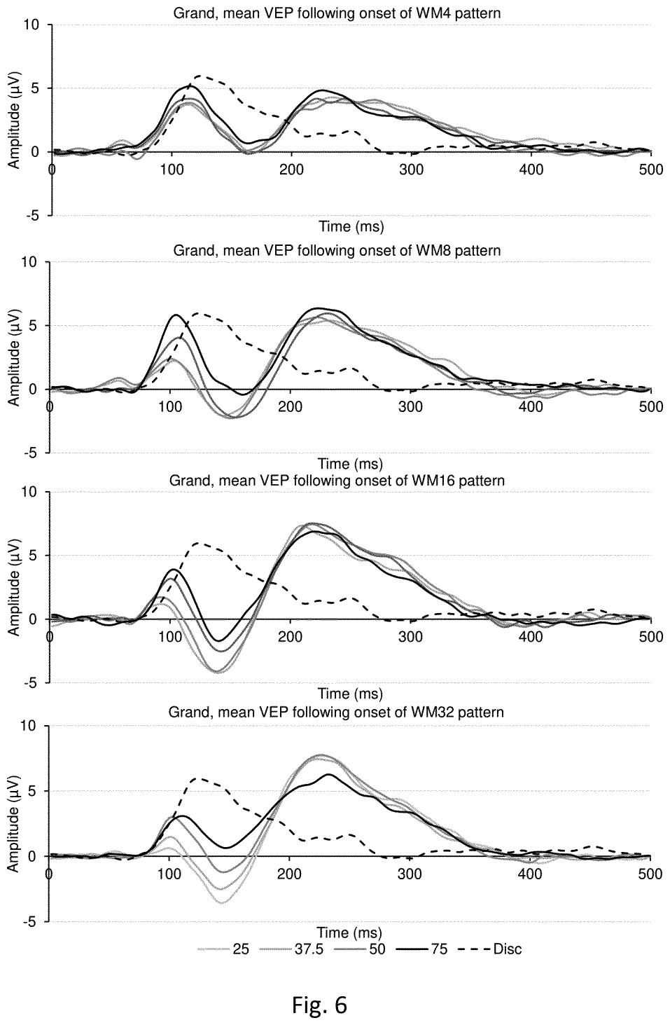

FIG. 6 shows grand mean VEP signals as a function of time, measured during the onset frames of the onset/offset paradigm for 4, 8, 16 and 32 white or black circular sectors as well as for fractions of 12.5%, 25%, 37.5% and 50% of the circle area being covered by white circular sectors, as well as a full white circle:

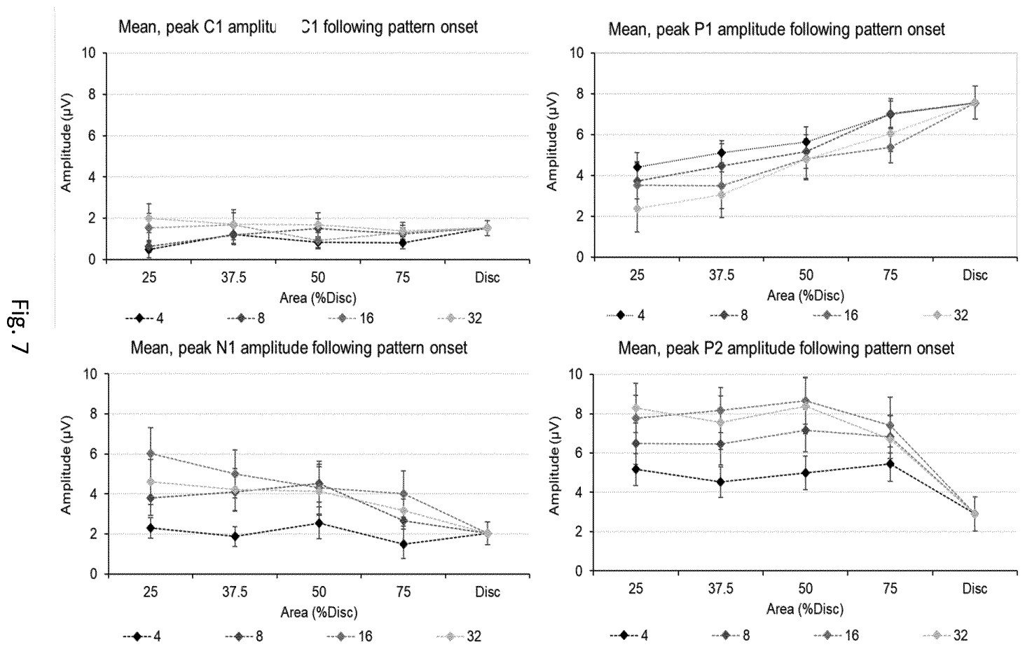

FIG. 7 shows the respective C1, P1, N1 and P2 absolute mean component amplitudes of the VEP signals shown in FIG. 6;

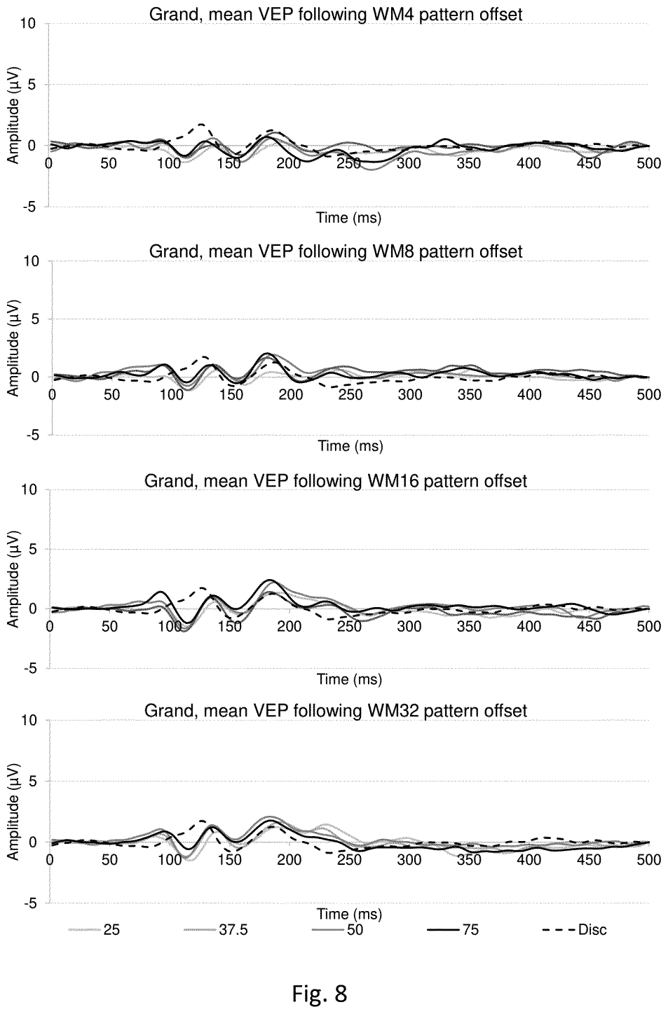

FIG. 8 shows grand mean VEP signals as a function of time, measured during the offset frames of the onset/offset paradigm for 4, 8, 16 and 32 white or black circular sectors as well as for fractions of 12.5%, 25%, 37.5% and 50% of the circle area being covered by white circular sectors;

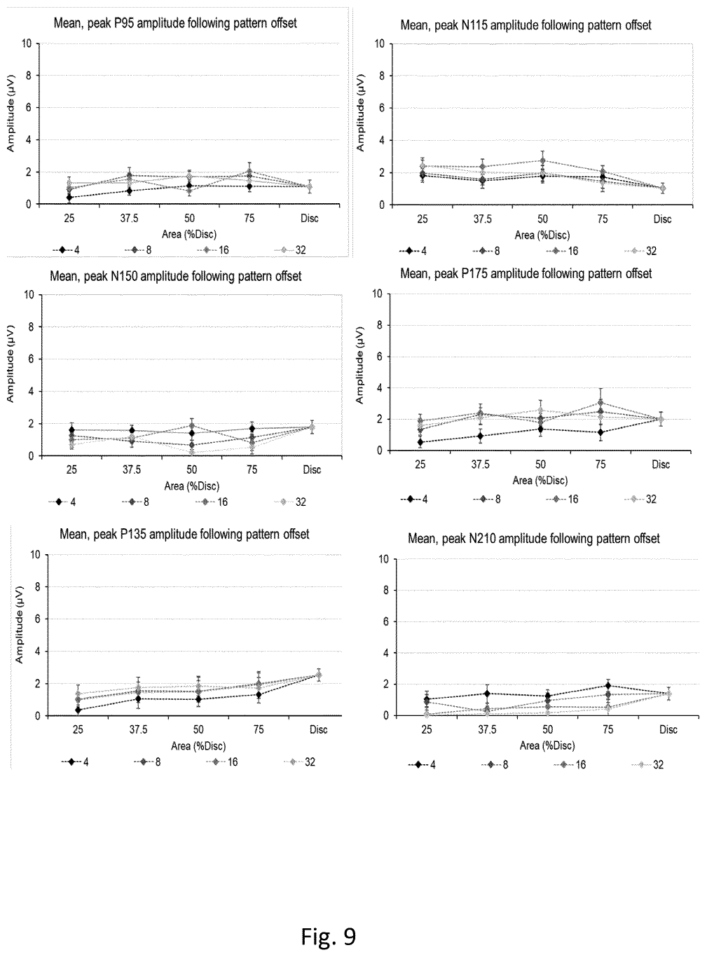

FIG. 9 shows the respective P95, N115, P135, N150, P175 and N 210 mean absolute component amplitudes of the VEP signals shown in FIG. 8;

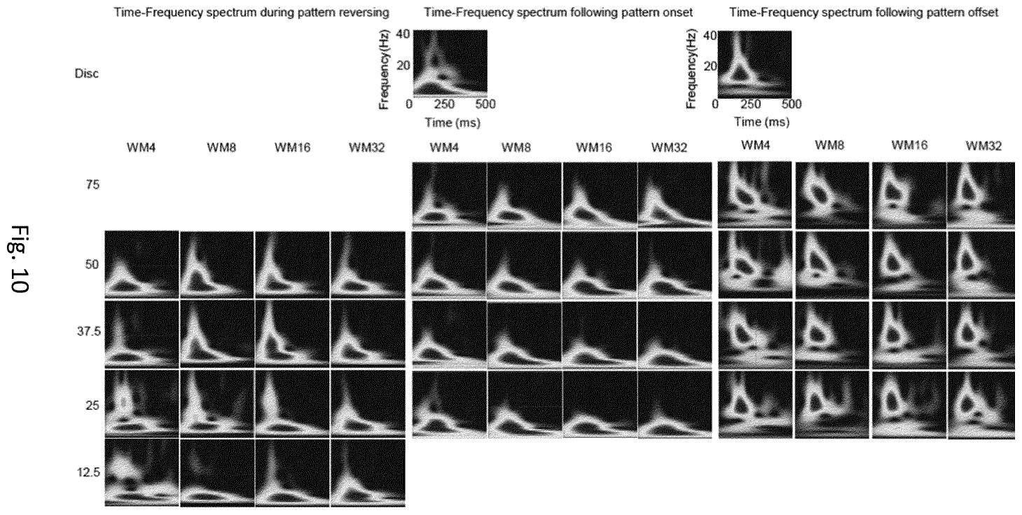

FIG. 10 shows time-frequency spectra of the VEP, measured using the pattern reversing paradigm as well during the onset and offset frames using the onset/offset paradigm;

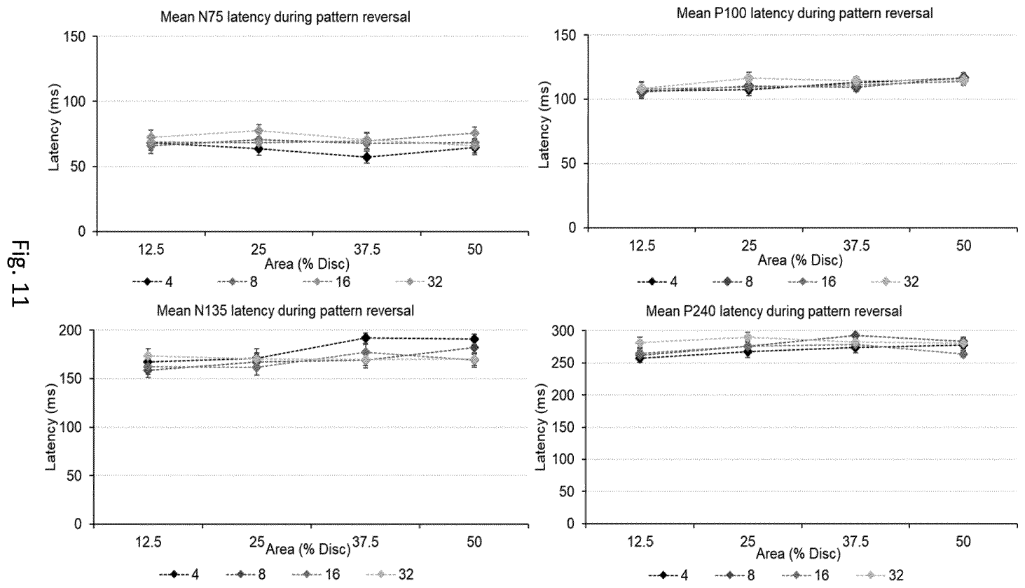

FIG. 11 shows the latency of the respective N75, P100, N135 and P240 component measured using the pattern reversing paradigm for 4, 8, 16 and 32 white or black circular sectors;

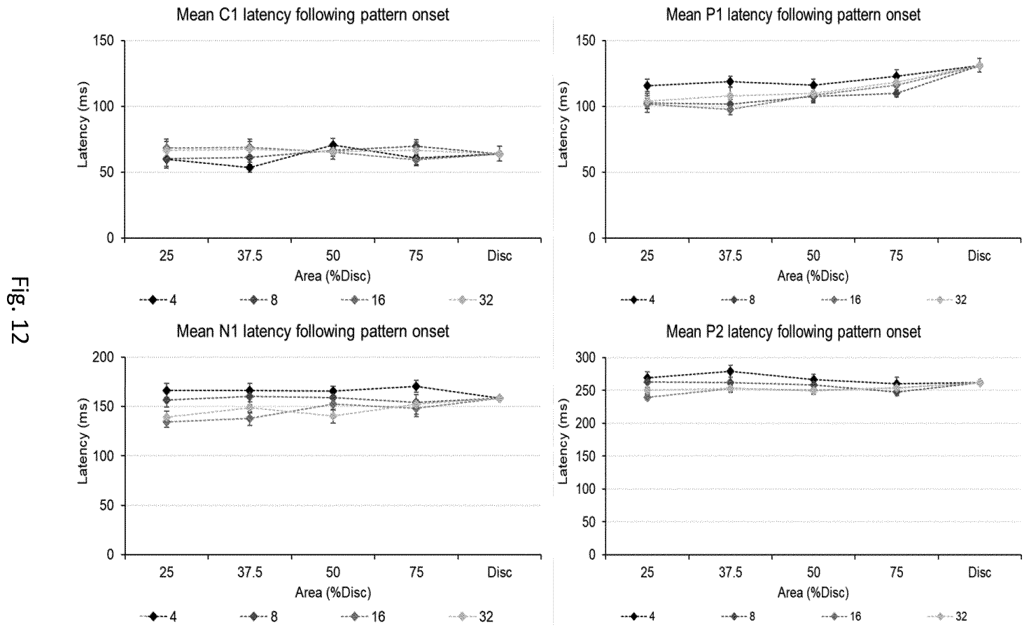

FIG. 12 shows the latency of the respective C1, P1, N1 and P2 component measured during the onset frames using the onset/offset paradigm; and

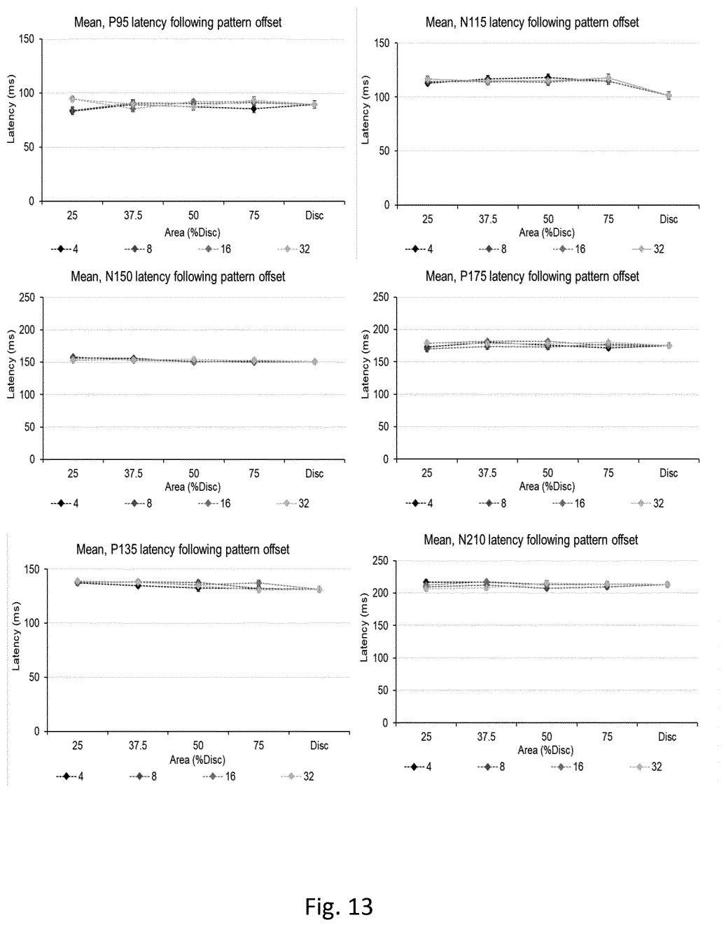

FIG. 13 shows the latency of the respective P95, N115, P135, N150, P175 and N210 component measured during the offset frames using the onset/offset paradigm.

FIG. 1 shows a schematic view of an embodiment of a medical system 20 according to the present invention for measuring visual evoked potentials of a person (e.g. for identifying a pathological change in a retina 10, particularly macula, of the person.

The medical system 20 comprises an image generating module 1, a display 2 (e.g. a computer monitor), a plurality of electrodes 3, an electroencephalography (EEG) recording module 4, optionally a camera 5 and an analyzing module 8. Preferably, the medical system 20 further comprises a chin rest 6 and/or a forehead rest 7 to fix a position of the person's head.

Particularly, the person places his/her head on said chin rest 6 and/or forehead rest 7 in a comfortable position during use of the medical system 20, such that the person is facing the display 2, as indicated in FIG. 1.

By using said chin rest 6 and/or said forehead rest 7, the influence of muscle artefacts in the measured visual evoked potential (VEP) is reduced.

For measuring a VEP signal according to the present invention, the patient is typically observing the display 2 that is being controlled by the image generating module 1 to show a sequence of a first and a second image in form of windmill patterns 30 that are alternately shown of a first image in form of a windmill pattern 30 that is turned on (onset) and off (offset).

The person is informed to fixate central point of the windmill patterns 30 with one eye (the other eye is covered) so that the images are properly projected onto the retina 10 of said eye of the person while the EEG recording module 4 is recording EEG signals induced in the brain of the patient and detected by the electrodes 3 in response to observing said sequence of images.

The EEG signals correspond to time-dependent voltages between electrodes 3. The EEG signals recorded by the EEG recording module 4 are preferably forwarded to and processed by the analyzing module 8 to derive the respective visual evoked potential (VEP) from the recorded EEG signals.

In the respective sequence of images (cf. e.g. FIG. 3) the first image is preferably projected for a predefined period of time, for example for 500 ms, and may for example comprise a windmill pattern 30 comprising light (e.g. white) or dark (e.g. black) circular sectors on the otherwise dark display surface of display 2. A subsequent second image may for example differ by inverting the contrast of the (light and dark) circular sectors, wherein these first and second images are each shown for e.g. 500 ms in an alternating fashion which is termed pattern alternating display or pattern reversing paradigm. Alternatively, the second image can be a fully dark (e.g. black) image. This is termed pattern on/off stimulus or pattern on/off paradigm.

As indicated in FIG. 1, for example, each light (e.g. white) circular sector 31 is delimited by two radii 31a extending from a common central point 33 in the radial direction and an arc 31b extending in a circumferential direction. Particularly, the respective image shown on display 2 comprises the respective pattern 30 and a region 35 adjacent a circle defined by the light and dark (e.g. black) circular sectors 31, 32 of the pattern 30, said region 35 having preferably the same color and luminance as the dark circular sectors 32, so that the dark circular sectors 32 seamlessly blend into said region 35 of the respective image shown on display 2.

The total sequence of images to be projected onto the retina 10 of the person may take for example 60 s. To obtain a good VEP signal, the VEP of each stimulus was obtained by averaging EEG segments of e.g. 500 ms duration starting at the marker marking the appearance of the stimulus (e.g. pattern onset of pattern offset in the respective sequence used). The averaged VEP signal for a respective pattern recorded for an individual patient may further be averaged using a group of patients, resulting in the grand mean VEP signal.

To demonstrate the invention, in an example, a group of fourteen healthy volunteers (8 ♀), aged between 23 yrs and 58 yrs and having normal vision, has been examined to obtain a grand mean VEP that can e.g. be used as a reference curve for identifying pathological changes in the retina/macula of a patient. Regarding the reference curve, at present a reference VEP from over 50 participants is available.

In the present example, for realizing the image generating module 1, the respective sequence of images has been programmed and controlled using Presentation (Version 20, NBS, Berkley, CA, USA) running on a desktop PC (HP Elite 7800) with a GeForce GTX 1050Ti graphics card running under Windows 10. The stimuli were presented on display 2 in form of a 28″ monitor (Brilliance 288P, Philips, Eindhoven, Netherland) using a refresh rate of 60 Hz. Its spatial resolution was set to 1900×1200 pixel, brightness to 80% and contrast to 90%. At the viewing distance of 1.0 m the pattern covered the central 15° of the visual field of the respective person. Muscle artefacts were minimized by having the respective person's head rest on a chin and forehead rest 7 (Richmond Products Inc., Albuquerque, NM, USA) when viewing the stimuli.

In the present example, EEG recording using the EEG recording module 4 followed the guidelines of the International Society for Clinical Electrophysiology of Vision (ISCEV) of 2009 (Odom et al., 2010). The scalp electric potential of the respective person was recorded using 32 active electrodes 3 located at the following sites (cf. also FIG. 1): Fp1/2, F7/8, F3/4, FC5/6, Fz, FC1/2, T7/8, C3/4, Cz, TP9/10, CP5/6, CP1/2, P7/8, P3/4, 20 Pz, PO9/10, O1/2 & Oz. This corresponded to the extended international 10/10 system (Chatrian, Lettich, & Nelson, 1985; Jasper, 1958). The ‘GROUND’ electrode was placed at AFz and the ‘REFERENCE’ electrode at FCz. Electrode impedance was kept at 5 kΩ or below and the sampling rate was set to 500 Hz. The EEG data was recorded and stored using commercial software (Brain Vision Recorder, Brain Products, Munich, 4 Germany, RRID: SCR_009443). During a recording session the room was in darkened, with the display 2 being the only source of illumination.

Furthermore, in the present example, the analyzing module 8 can be realized by means of a software being executed on a computer 9. Particularly, the recorded EEG signals have been analyzed using Analyzer Version 2.1 (BrainVision, Munich, Germany, RRID: SCR_002356). The EEG data was bandpass filtered so that oscillations below 0.5 Hz and above 40 Hz were eliminated, as were signal changes with a slope lower than 24 dB/oct and higher 48 dB/oct. Artefacts due to eye blinks were identified using an independent component analysis and removed. Any remaining artefacts, i.e. due to muscle movements, were identified by visual inspection and removed. Further, the signal from each electrode was re-referenced against the mean signal of all electrodes 3. The VEP of each stimulus was obtained by averaging EEG segments of 500 ms duration starting at the marker marking the appearance of the stimulus. For each VEP the amplitude and latency of four VEP components have been calculated: N75/C1, P100/P1, N135/N1 and P240/P2.

For the VEP from the pattern alternating display the minimum between 50 ms and 95 ms was taken as N75 amplitude. The maximum between 70 ms and 140 ms was taken as P100 amplitude. The minimum between 100 ms and 150 ms was taken as N135 amplitude. The maximum between 180 ms and 300 ms was takes as the P240 amplitude.

For the VEP following pattern onset the minimum between 50 ms and 95 ms was taken as C1 amplitude. The maximum between 70 ms and 140 ms was taken as P1 amplitude. The minimum between 100 ms and 150 ms was taken as N1 amplitude. The maximum between 180 ms and 300 ms was takes as the P2 amplitude.

The appearance of the VEP following pattern offset differed substantially in appearance to that following onset of the pattern. Six deflections have been identified alternating between positive and negative electric polarity. Their respective peak occurred at 95 ms, 115 ms, 135 ms, 150 ms, 175 ms and 210 ms. We termed the six components P95, N115, P135, N150, P175 and N210 reflecting the electric polarity and time at which they peaked. The maximum between 70 ms and 100 ms was taken as the amplitude of P95, the minimum between 95 ms and 114 ms was taken as the amplitude of N115, the maximum between 100 ms and 140 ms as the amplitude of P135, the minimum between 120 ms and 170 ms as the amplitude of N150, the maximum between 160 ms and 190 ms as the amplitude of P175 and the minimum between 190 ms and 230 ms as the amplitude of N210. The time of the maximum or minimum served as the latency of a VEP component.

Furthermore, the amplitude and latency of the four VEP components to the pattern alternating display and following on-/offset of the pattern were compared using a multi-factorial ANOVA with repeated measures as implemented in JASP (Version 0.14.0, JASP Team (2020)). For the other comparison of VEP amplitude and latency, “SECTORS” and “AREA” were the repeated measures factors. Previously, it has been demonstrated that N75 and C1 and P100 and P1 arise from temporal luminance contrast processing and N135 and N1 and P240 and P2 from spatial luminance contrast processing (Marcar et al., 2018; Marcar & Jancke, 2016, 2018). Following this approach, the amplitude and latency of P100 vs P1 and P240 vs P2 components has been compared to identical windmills recorded during a pattern alternating display and following their onset. “MODE” represented the between subject factor and “SECTORS” and “AREA” the repeated measures factors. To reduce the risk of a Type I error p≤0.01 was accepted as indicating a significant difference.

Furthermore, according to an embodiment of the medical system 20 according to the present invention, the system 20 can further comprise a camera 5 operatively connected to the image generating module 1, the camera 5 being configured to track eye movements of the person (using e.g. a tracking algorithm) while a sequence of windmill patterns 30 is projected onto the retina 10 of the person via the display 2. Particularly, said tracking algorithm is configured to detect whether the person's eye being examined is focusing on the central point of the windmill patterns 30, wherein in case the person is not fixating said central point the image generating module 1 does not cause the display to display an image of the sequence.

Furthermore, FIG. 2 shows various exemplary windmill patterns 30 to be projected onto a retina 10 of a patient via the monitor for inducing VEP in the person's brain according to the invention. To achieve a significant VEP signal, windmill patterns 30 with different numbers of light (e.g. white) and dark (e.g. black) circular sectors and/or different fractions of the circle area covered by the light circular sectors 31 may be programmed by the image generating module 1 and projected by the display 2 controlled by the image generating module 1. As shown in FIG. 2, the respective sequences of projected images may have different numbers of light and dark circular sectors 31,32 and/or different fractions of the circle area being covered by light circular sectors 31. Preferably, as shown in FIG. 2, the number of light (and dark) circular sectors is one of: 4,8,16, 32 with fractions of 12.5%, 25%, 37.5% or 50% of the circle area being covered by light circular sectors 31. Every combination is possible. The most preferred embodiment being 4 light (e.g. white) circular sectors and 4 dark (e.g. black) circular sectors with the light circular sectors 31 making up 50% of the area of the circle.

According to an embodiment of the present invention, the projected images comprising the circle and the circular sectors have a resolution of 1200×1200 pixels, with an exemplary luminance of 167 cd/m² of the light (white) sectors and an exemplary luminance of 0.015 cd/m² of the dark (black) sectors. The Michelson contrast between said light and dark sectors may for example be set to 99.8.

FIG. 3 schematically shows sequences of an exemplary windmill pattern 30 using the pattern reversing paradigm as well as the onset/offset paradigm programmed by the image generating module 1 and projected onto a retina 10 of a patient via a monitor 2 controlled by the image generating module 1.

The windmill pattern 30 used for the respective sequences may for example be a windmill pattern 30 comprising each 8 black and white circular sectors, where the fraction of the circle area covered by white circular sectors with respect to the total circle area is for example set to 25%, as shown in FIG. 3.

According to an embodiment of the present invention, using the pattern reversing paradigm, the VEP is continuously recorded while projecting an alternating sequence of a first frame comprising a respective windmill pattern 30 with a given number of black and white circular sectors as well as a given fraction of the circle area covered by white circular sectors with respect to the total circle area onto a retina 10 of a patient, as well as a second frame comprising the respective windmill pattern 30 with the black and white areas being replaced by white and black areas, respectively. The individual frames are preferably projected for 500 ms with a total sequence time of for example 60 s, as indicated in FIG. 3 (top). According to another embodiment of the present invention, using the pattern on/-offset paradigm, the VEP is continuously recorded while projecting a sequence of a first frame comprising a windmill pattern 30 with a given number of black and white circular sectors as well as a given fraction of the circle area covered by white circular sectors with respect to the total circle area followed by a second frame comprising a black screen. The individual frames are preferably projected for 500 ms with a total sequence time of for example 60 s, as indicated in FIG. 3 (bottom).

FIG. 4 shows grand mean VEP signals as a function of time, measured using the pattern reversing paradigm for 4, 8, 16 and 32 white or black circular sectors as well as for fractions of 12.5%, 25%, 37.5% and 50% of the circle area being covered by white circular sectors. As FIG. 4 demonstrates, a larger number of circular sectors gives rise to a systematic decrease of the P100 amplitude as well as an increase of the N75, N135 and P240 amplitudes.

FIG. 5 shows the respective N75, P100, N135 and P240 absolute mean component amplitudes of the VEP signals shown in FIG. 4. While the N75 amplitude does not show a dependence on the fraction of the circle area covered by white circular sectors, it systematically increases with the numbers of black or white sectors. The P100 amplitude shows a linear dependence on both the number of black or white circular sectors and the fraction of the circle area covered by white circular sectors.

The invention deviates from the present practice of using a pattern in which the white and black circular sectors occupy 50% of the area, respectively, and/or in which the black and the white circular sectors are exchanged in every consecutive frame. In contrast, according to the invention, the second image corresponds to the first image, but with the order of the light circular sectors and the dark circular sectors being reversed.

This pattern alternating display or pattern reversing paradigm (introduced in FIG. 3) has been applied for the measurements presented in FIG. 5. In these measurements, sequences of images with the first and the second image displayed according to the pattern alternating display have been displayed for different percentages of the area of the circle covered by white circular segments and for different numbers of equidistantly spaced dark circular sectors.

The retinotopic organisation of primary visual cortex has adjacent points in the retina occupy adjacent points in the primary visual cortex. There is thus a direct correspondence between the area of the retina activated by the elements of a pattern and the activated area in the primary visual cortex. Furthermore, there is a direct relationship between size of active area in the primary visual cortex and the number of active neurons in the primary visual cortex.

This dependence can be seen by the linear dependence of the P100 amplitude as a function of the percentage of the area of the circle, which occurred independently of the of number of equidistantly spaced white and dark circular sectors.

As such, the invention represents a means of assessing the number of active receptors in the retina from the visual evoked potential obtained over the occipital pole and so a means to determine retinal health.

Furthermore, Table 1 contains the results of the analysis of variance of the amplitude of the four VEP components to the percentage of the stimulus area undergoing a change in luminance contrast and the number of sectors over which this change was spread. N75 amplitude was unaffected by the total stimulus area undergoing a change in luminance contrast but increases as the number of sectors over which this area was distributed increased. P100 amplitude increased linearly with the size of the area undergoing a change in luminance contrast but was unaffected by the number of sectors over which this area was distributed. The factors SECTORS and AREA were found to interact, with a larger number of sectors leading to a stronger attenuation of P100 amplitude at larger total area undergoing a change in luminance contrast. N135 amplitude did not signal stimulus area undergoing a change in luminance contrast but increased as the number of sectors over which this area was distributed increased. P240 amplitude did not reflect stimulus area undergoing a change in luminance contrast nor the number of sectors over which this area was distributed. The two factors were found to interact, with an increase in the number of sectors in the image attenuating the influence of area undergoing a change in luminance contrast.

TABLE 1
The table contains the results of the multi-factorial analysis
of variance of amplitude of each VEP component during pattern
reversal, with size of stimulus area undergoing a change
luminance contrast and the number sectors over which this
area was distributed as repeated measures factors.

| Within-subject-effect | F | Hypothesis df | Error df | p | η² |
|---|---|---|---|---|---|
| N75 | | | | | |
| SECTORS | 3.753 | 2.131 | 27.705 | .034 | .045 |
| AREA | .661 | 2.181 | 28.352 | .537 | .004 |
| SECTORS*AREA | 1.01 | 2.925 | 38.025 | .397 | .018 |
| P100 | | | | | |
| SECTORS | 1.91 | 2.633 | 34.229 | .153 | .018 |
| AREA | 41.317 | 2.353 | 3.588 | 10⁻³ | .359 |
| SECTORS*AREA | 3.797 | 7.333 | 95.335 | 10⁻³ | .034 |
| N135 | | | | | |
| SECTORS | 12.383 | 2.010 | 26.124 | 10⁻³ | .123 |
| AREA | .520 | 2.412 | 31.356 | .633 | .002 |
| SECTORS*AREA | .934 | 7.284 | 94.686 | .487 | .009 |
| P240 | | | | | |
| SECTORS | 5.104 | 2.196 | 28.548 | .011 | .055 |
| AREA | 2.522 | 1.915 | 24.899 | .103 | .011 |
| SECTORS*AREA | 5.214 | 8.799 | 114.389 | 10⁻³ | .049 |

Furthermore, the four panels of FIG. 6 show the grand, mean VEP following pattern onset. The number of circular sectors in the image doubles from top to bottom. The most prominent changes in the appearance of the VEP are: 1) that it is simpler in structure following onset of the disc than following onset of the windmill pattern 30, 2) P1 amplitude increases and N1 amplitude decreases as the stimulus area undergoing a change in luminance contrast increases, 3) P1 amplitude decreases and N1 and P2 amplitude increase as the number of circular sectors over which the area undergoing a change in luminance contrast increases. While P1 amplitude decreased, N1 and P2 amplitude increased as the number of sectors in the display increased.

Further, the four panels of FIG. 7 depict the mean, peak amplitude C1, P1, N1 and P2 in the VEP components following pattern onset.

Particularly, Table 2 contains the results of the analysis of variance of the amplitude of the four VEP components to the percentage of the stimulus area undergoing a change in luminance contrast and the number of sectors over which this change was spread. C1 amplitude varied neither with stimulus area undergoing a change in luminance contrast nor with the number of sectors over which this area was distributed across the image. P1 amplitude increased as the total stimulus area undergoing a change in luminance contrast increased. Its amplitude decreased as the number of sectors over which this area was distributed increased. The VEP following onset of the disc lacked deflections beyond P1 so only the VEP following onset of the windmill pattern 30 will be reported for N1 and P1. N1 amplitude decreased as the total stimulus area undergoing a change in luminance contrast increased but increased as the number of sectors over which this area was distributed increased. P2 amplitude was unaffected by the size of the area undergoing a change in luminance contrast but increased as the number of sectors over which this area was distributed increased.

TABLE 2
The table contains the results of the multi-factorial analysis
of variance of amplitude of each VEP component following
pattern onset, with size of stimulus area undergoing a change
luminance contrast and the number sectors over which this
area was distributed as repeated measures factors.

| Within-subject-effect | F | Hypothesis df | Error df | p | η² |
|---|---|---|---|---|---|
| C1 | | | | | |
| SECTORS | 4.244 | 2.595 | 33.737 | .015 | .031 |
| AREA | .586 | 2.731 | 35.506 | .613 | .004 |
| SECTORS*AREA | .785 | 4.580 | 59.543 | .555 | .020 |
| P1 | | | | | |
| SECTORS | 3.613 | 2.419 | 31.447 | .031 | .028 |
| AREA | 17.001 | 2.889 | 37.554 | 10⁻³ | .095 |
| SECTORS*AREA | .544 | 7.047 | 91.612 | .800 | .007 |
| N1 | | | | | |
| SECTORS | 5.315 | 2.105 | 27.363 | .010 | .070 |
| AREA | 3.465 | 3.000 | 39.000 | .025 | .017 |
| SECTORS*AREA | .633 | 5.183 | 67.377 | .681 | .009 |
| P2 | | | | | |
| SECTORS | 11.674 | 1.926 | 25.043 | 10⁻³ | .075 |
| AREA | .369 | 2.654 | 34.503 | .369 | .004 |
| SECTORS*AREA | .594 | 4.999 | 64.993 | .594 | .008 |

Furthermore, the four panels of FIG. 8 show the grand, mean VEP following pattern offset. The number of sectors in the pattern doubles from top to bottom. The timing of the deflections in the VEP following offset of the pattern differs substantially from that observed in the VEP following onset of the pattern. However, the similarity between the VEP to the different pattern indicates that the same set of neural responses ensues after offset of the pattern.

FIG. 9 shows the respective P95, N115, P135, N150, P175 and N 210 mean absolute component amplitudes of the VEP signals shown in FIG. 8. None of the VEP components reacted to changes in the fraction of the circle area being covered by white circular sectors. In contrast, the amplitudes of the components P95, N150 and P175 increased with the number of black or white circular sectors, while the amplitude the N210 component decreased.

Further, Table 3 contains the results of the analysis of variance of the amplitude of the four VEP components to the percentage of the stimulus area undergoing a change in luminance contrast and the number of sectors over which this change was spread. None of the components reacted to size of the stimulus area undergoing a change in luminance contrast. P95, N150, and P175 amplitude increased as the number of sectors over which this area was distributed increased. In contrast N210 amplitude decreased as the number of sectors in over which this area was distributed increased.

TABLE 3
The table contains the results of the multi-factorial analysis
of variance of amplitude of each VEP component following
pattern offset, with size of stimulus area undergoing a change
luminance contrast and the number sectors over which this
area was distributed as repeated measures factors

| Within-subject-effect | F | Hypothesis df | Error df | p | η² |
|---|---|---|---|---|---|
| P95 | | | | | |
| SECTORS | 5.005 | 3.000 | 39.000 | .005 | .033 |
| AREA | 3.026 | 2.107 | 27.395 | .063 | .030 |
| SECTORS*AREA | 1.264 | 6.162 | 80.108 | .282 | .028 |
| N115 | | | | | |
| SECTORS | 3.718 | 3.000 | 39.000 | .019 | .027 |
| AREA | 1.465 | 3.000 | 39.000 | .239 | .015 |
| SECTORS*AREA | .544 | 5.808 | 75.498 | .767 | .011 |
| P135 | | | | | |
| SECTORS | 2.143 | 2.877 | 37.404 | .113 | .015 |
| AREA | 2.326 | 1.988 | 25.849 | .118 | .017 |
| SECTORS*AREA | .133 | 5.140 | 66.823 | .985 | .002 |
| N150 | | | | | |
| SECTORS | 4.787 | 3.000 | 39.000 | .006 | .048 |
| AREA | .195 | 3.000 | 39.000 | .899 | .002 |
| SECTORS*AREA | 1.517 | 7.618 | 99.033 | .164 | .038 |
| P175 | | | | | |
| SECTORS | 12.318 | 2.383 | 3.975 | 10⁻³ | .059 |
| AREA | 2.456 | 1.748 | 22.718 | .114 | .023 |
| SECTORS*AREA | .864 | 7.248 | 94.226 | .541 | .014 |
| N210 | | | | | |
| SECTORS | 8.672 | 1.790 | 23.271 | .002 | .070 |
| AREA | 2.348 | 2.657 | 34.539 | .096 | .021 |
| SECTORS*AREA | .383 | 8.465 | 11.044 | .934 | .008 |

No difference was reported in amplitude between P100 and P1 but a substantial difference between P240 and P2 to the identical dartboard images viewed during a pattern alternating display and following dartboard onset (Marcar & Jancke, 21 2016). The present analysis of the amplitude of these components also revealed no difference between P100 and P1 (F=0.004, df=1,26, p=0.984, η²=0.000) but a substantial difference between P240 and P2 (F=29.164, df=1,26, p<10⁻³, η²=0.529).

FIG. 10 depicts the Winger plots from the VEP to a pattern alternating display are characterized by the strong presence of oscillations in the Θ-band (4-8 Hz), a decrease in prominence of oscillations in the upper β-band (20-30 Hz) as the number of sectors increases but an increase prominence of oscillations in the lower β-band (12-20 Hz) with increasing area undergoing a change in luminance contrast. The VEP following pattern onset is characterized by prominence of oscillations in the Θ-band (4-8 Hz). Compared to the VEP obtained during a pattern alternating display, oscillations in this frequency band persist for longer, until offset as the number sectors increased. The VEP following pattern offset is characterized by prominence of oscillations in the β-band (12-30 Hz) that subside quickly.

FIG. 11 shows the latency of the respective N75, P100, N135 and P240 VEP component measured using the pattern reversing paradigm for 4, 8, 16 and 32 white or black circular sectors.

Furthermore, Table 4 contains the results of the analysis of variance of the latencies of these four VEP components with area undergoing a change in luminance contrast and the number of sectors over which this area was distributed.

TABLE 4
The table contains the results of the multi-factorial analysis
of variance of latency of each VEP component during pattern
reversal, with size of stimulus area undergoing a change
luminance contrast and the number sectors over which this
area was distributed as repeated measures factors.

| Within-subject-effect | F | Hypothesis df | Error df | p | η² |
|---|---|---|---|---|---|
| N75 | | | | | |
| SECTORS | 2.569 | 2.843 | 36.956 | 0.072 | 0.024 |
| AREA | 0.298 | 2.804 | 36.454 | 0.814 | 0.004 |
| SECTORS*AREA | 1.015 | 9.000 | 117.000 | 0.432 | 0.022 |
| P100 | | | | | |
| SECTORS | 0.669 | 2.349 | 30.54 | 0.543 | 0.006 |
| AREA | 2.175 | 2.047 | 26.614 | 0.132 | 0.034 |
| SECTORS*AREA | 1.045 | 6.223 | 80.900 | 0.403 | 0.010 |
| N135 | | | | | |
| SECTORS | 3.531 | 1.918 | 24.934 | 0.046 | 0.029 |
| AREA | 8.458 | 2.147 | 27.91 | 0.001 | 0.039 |
| SECTORS*AREA | 3.005 | 6.101 | 79.313 | 0.010 | 0.034 |
| P240 | | | | | |
| SECTORS | 2.745 | 1.618 | 21.04 | 0.096 | 0.036 |
| AREA | 4.035 | 2.570 | 33.413 | 0.019 | 0.034 |
| SECTORS*AREA | 1.292 | 6.051 | 78.657 | 0.270 | 0.030 |

The N75, P100 and P240 latencies were unaffected by the total stimulus area undergoing a change in luminance contrast or the number of sectors over which this area was distributed. N135 increased with the size of the area undergoing but decreased with the number of sectors over which this area is distributed.

FIG. 12 depicts the latencies of C1, P1, N1 and P2 VEP components following onset of an image. Furthermore, Table 5 contains the results of the analysis of variance of the latencies of these four VEP components with area undergoing a change in luminance contrast and the number of sectors over which this area was distributed.

TABLE 5
The table contains the results of the multi-factorial analysis
of variance of latency of each VEP component following
pattern onset with size of stimulus area undergoing a change
luminance contrast and the number sectors over which this
area was distributed as repeated measures factors.

| Within-subject-effect | F | Hypothesis df | Error df | p | η² |
|---|---|---|---|---|---|
| C1 | | | | | |
| SECTORS | 1.234 | 3.000 | 39.000 | 0.31 | 0.010 |
| AREA | 0.896 | 3.000 | 39.000 | 0.452 | 0.009 |
| SECTORS*AREA | 1.037 | 7.507 | 97.590 | 0.412 | 0.031 |
| P1 | | | | | |
| SECTORS | 2.964 | 2.791 | 36.286 | 0.048 | 0.073 |
| AREA | 6.922 | 2.65 | 34.448 | 0.001 | 0.047 |
| SECTORS*AREA | 1.106 | 6.795 | 88.335 | 0.366 | 0.016 |
| N1 | | | | | |
| SECTORS | 9.148 | 2.697 | 35.056 | 10⁻³ | 0.123 |
| AREA | 1.322 | 2.742 | 35.650 | 0.282 | 0.014 |
| SECTORS*AREA | 0.898 | 7.797 | 101.356 | 0.519 | 0.018 |
| P2 | | | | | |
| SECTORS | 6.048 | 2.500 | 32.496 | 0.003 | 0.071 |
| AREA | 1.351 | 2.665 | 34.644 | 0.274 | 0.008 |
| SECTORS*AREA | 1.149 | 6.219 | 80.844 | 0.342 | 0.030 |

P1 latency increased as the size of the area undergoing a change in luminance contrast. N1 and P2 latency increased as the number of sectors over which this area was distributed increased.

Further, FIG. 13 depicts the latencies of P95, N115, P135, N150, P175 and N210 following offset of an image. Particularly, the latency of none of the VEP components examined was influenced by size of the area undergoing a change in luminance contrast. Only N210 was found to shorten as the number of sectors over which this area was distributed increased.

TABLE 6
The table contains the results of the multi-factorial analysis
of variance of latency of each VEP component following pattern
offset, with size of stimulus area undergoing a change luminance
contrast and the number sectors over which this area was
distributed as repeated measures factors.

| Within-subject-effect | F | Hypothesis df | Error df | p | η² |
|---|---|---|---|---|---|
| P95 | | | | | |
| SECTORS | 2.144 | 3.000 | 39.000 | .110 | .033 |
| AREA | .410 | 2.038 | 26.494 | .671 | .003 |
| SECTORS*AREA | 2.312 | 7.926 | 103.034 | .026 | .069 |
| N115 | | | | | |
| SECTORS | .079 | 2.856 | 37.128 | .967 | .001 |
| AREA | .232 | 2.810 | 36.526 | .862 | .002 |
| SECTORS*AREA | .797 | 3.518 | 45.738 | .520 | .018 |
| P135 | | | | | |
| SECTORS | 2.458 | 3.000 | 39.000 | .077 | .017 |
| AREA | 2.924 | 1.390 | 18.074 | .094 | .051 |
| SECTORS*AREA | 2.924 | 5.602 | 72.828 | .473 | .024 |
| N150 | | | | | |
| SECTORS | .116 | 2.029 | 26.382 | .893 | .001 |
| AREA | 1.670 | 2.711 | 35.245 | .195 | .021 |
| SECTORS*AREA | .756 | 6.301 | 81.916 | .612 | .019 |
| P175 | | | | | |
| SECTORS | 3.396 | 2.365 | 3.74 | .039 | .028 |
| AREA | .547 | 2.191 | 28.484 | .600 | .006 |
| SECTORS*AREA | .915 | 7.363 | 95.717 | .502 | .026 |
| N210 | | | | | |
| SECTORS | 4.429 | 3.000 | 39.000 | .009 | .030 |
| AREA | .282 | 2.531 | 32.899 | .805 | .003 |
| SECTORS*AREA | 1.019 | 6.358 | 82.66 | .421 | .028 |

In the above describe example, the VEP following onset of the disc is simpler in structure than the VEP following onset of a windmill pattern 30. N75 and C1 amplitude showed no reaction to neither the change in relative stimulus area occupied by white nor the number of sectors over which this area was distributed. P100 and P1 amplitude increased with the relative stimulus area occupied by white but their amplitude decreased as the number of sectors in the pattern increased. For the identical pattern, P100 during a pattern alternating display and P1 following pattern onset were equal in amplitude. N135 and N1 as well as P240 and P2 amplitude did not react to the relative stimulus area occupied by white but increased with the number of sectors over which the area occupied by white increased. P2 amplitude following pattern onset exceeded P240 amplitude to the same pattern viewed as a pattern alternating display. Deflections in the VEP following offset of a pattern occurred at different times than those observed following onset of the pattern. Its components did not react to the relative stimulus area occupied by white pixels and only the initial component and final three components responded to the number of sectors in the pattern.

Only N135 latency increased as the number of sectors in the pattern increased. For the same pattern, N1 and P1 latency were shorter than N75 and P100 latency. P1 latency increased as the relative stimulus area occupied by white increased. N1 and P2 latency decreased as the number of sectors in the pattern increased.

The most apparent differences in the time-frequency spectrum of the VEP obtained following on- and offset of a pattern is the that oscillations in the Θ- and α-band are strongest in the VEP following pattern onset while oscillations in the β-band are strongest following offset of the pattern. While oscillations in the α- and β-band subside quickly, oscillations in the Θ-band also persist. α-band oscillations become more prominent as the area undergoing a change in luminance contrast increases. The latency of the last components in the VEP following offset of a pattern decreased as the number of sectors in the pattern increased.

Anatomically, the primate visual system is divided into striate and extra-striate cortex: the latter containing a multitude of different cortical areas (DeYoe et al., 1994). These areas form a dorsal and ventral processing stream (Ungerleider & Mishkin, 1982) acting in parallel. The former terminates in parietal lobe, the latter in inferior temporal lobe. Areas of the dorsal stream contain a retinotopic representation of at least part of the contralateral visual field (Brewer, Press, Logothetis & Wandell, 2002), while in areas of the ventral stream the retinotopic representation is coarse, with receptive field covering up to ⅓rd 10 of the contralateral visual space (Op De Beeck & Vogels, 2000) and gives way to a feature or object based representation (Grill Spector, Kourtzi, & Kanwisher, 2001; Orban, Zhu, & Vanduffel, 2014; Tanaka, 1993, 1996). and position invariance (Anzellotti & Caramazza, 2014; Booth & Rolls, 1998; Logothetis & Sheinberg, 1996; Perrett, Hietanen, Oram, & Benson, 1992; Rust & Dicarlo, 2010).

Functionally, the primate visual system processes three types of retinal signals carried by magno- parvo- and koniocellular neurons (Callaway, 1998; Hendry & Reid, 2000; Nassi & Callaway, 2006). Only the former two will be discussed here as koniocellular neurons are exclusively selective to chromatic contrast, rendering their contribution to the VEP recorded our achromatic stimuli negligible (Gouras, Mackay, & Yamamoto, 1993). Magnocellular neurons carry a temporal luminance contrast (dL/dt) signal, parvocellular neurons spatial luminance contrast (dL/ds) to striate cortex (Derrington & Lennie, 1984; Robson, 1966). Axonal conduction velocity of magnocellular neurons exceeds that of their parvocellular counterpart so that their signal activates neurons in striate cortex 50 ms after leaving the retina 10 (Foxe & Simpson, 2002), 20 ms ahead of the parvocellular signal (Klistorner et al., 1997; Laycock, Crewther, & Crewther, 2007). The number of parvocellular axons entering striate cortex exceeds the number of magnocellular axons by between ten (Ahmad & Spear, 1993) and forty fold (Azzopardi & Cowey, 1996). Magnocellular neurons enter striate cortex via the granular lamina IVCα, from where their signal proceed to lamina IVB and then to areas in extra-striate cortex. Parvocellular neurons enter the granular layer IVCβ from where their signal activated neurons in the supragranular laminae II/III followed by neurons in infragranular laminae V/VI before innervating areas in extra-striate cortex (Maunsell & Gibson, 1992).

The transient response of magnocellular—and sustained response of parvocellularneurons has been called into question (Blakemore & Vital-Durand, 1986; Skottun, 2014; Skottun & Skoyles, 2017) as has the view that the most magnocellular neurons project to areas of the dorsal processing stream and all parvocellular neurons project to areas of the ventral processing stream (Merigan & Maunsell, 1993; Skottun, 2015). Retinal signal processing in the primate visual system proceeds in parallel (DeYoe et al., 1994) and involves iterative interactions between cortical areas (Douglas & Martin, 2007). Reentrant projections avoid the granular input laminae preferentially terminating in the supra- and infragranular laminae (Lund, Angelucci, & Bressloff, 2003). Reentrant projections between area V2 and striate cortex of the monkey modulate the neural response driven by thalamic input within 8 ms (Hupe et al., 2001), which in the human visual system corresponds to 14 ms (Schroeder, Mehta, & Givre, 1998). Consequently, any neural response captured by the VEP has been modulated by reentrant projections.

In the following the contribution of magno- and parvocellular system to the neural response in the visual system is considered by examining the VEP for characteristics reflecting properties of magno- and parvocellular neurons. Interpolating single cell properties from characteristics of the VEP is evidently a challenging undertaking. There are a number of premises which indicate that reduce the risk of misinterpretation in the approach taken. Those pertinent to our approach are given below.

Psychophysical evidence points to the presence of a transient and sustained processing channel in the human visual system (Horiguchi, Nakadomari, Misaki, & Wandell, 2009; Ikeda & Wright, 1972; Kulikowski & Tolhurst, 1973; Tolhurst, 1975). The VEP arises from the neural response driven by mechanisms selective to temporal—and spatial luminance contrast (Marcar et al., 2018; Marcar & Jancke, 2016, 2018). Temporal luminance contrast selectivity is associated with magnocellular, spatial luminance contrast selectivity with parvocellular neurons (Derrington & Lennie, 1984; Robson, 1966). The VEP components signal the change in activity of all neurons responding, rather than the change in activity of a specific population (Mitzdorf et al., 1994). By focusing on the signal from electrode Oz, we were able to use the waxing and waning of the neural response in the same patch of striate cortex to the neural source of reentrant projections modulating the ongoing neural activity.

Particularly, P100 and P1 amplitude during temporal luminance contrast processing of a pattern viewed during an alternating display and following onset of the identical pattern were on par (See Graphs 4 & 6 and Table 4). For this to be the case the underlying neural response must be transient in nature, i.e. subside before the next image exchange. P100 and P1 amplitude matched the size of the neural population active during temporal luminance contrast processing, the stimulus property to which magnocellular neurons are selective (Derrington & Lennie, 1984; Robson, 1966). For this to be the case in spite of modulation of the neural response by re-entrant projections, these projections must conserve the spatial distribution of the thalamic activity. Reentrant projections between cortical areas with a retinotopic organisation are less diffuse than those between areas lacking such an organisation (Blasdel, Lund, & Fitzpatrick, 1985). This points to areas of the dorsal processing stream as the origin of reentrant projections that modulate the neural response in striate cortex during temporal luminance contrast processing.

This view is supported by the prominence of oscillation in the VEP with frequencies in the β-range during temporal luminance contrast processing. Oscillations in the VEP are the result of fluctuations in neural activity due to modulation of by interactions between cortical areas. Activation latencies of visual areas comprising the dorsal processing stream are shorter than those of their counterparts comprising the ventral processing stream (Chen et al., 2007). Feedback connections exhibiting magnocellular properties, that is, fast conduction velocity and low spatial resolution play an important role in the way information is processed within visual cortex and hence its activity during visual object processing (Bar, 2003; Kveraga, Boshyan, & Bar, 2007). A magnocellular role in the neural response during temporal luminance contrast processing also accounts for the observation that low spatial frequency processing is associated with high temporal frequency oscillations in the VEP (Frund, Busch, Korner, Schadow, & Herrmann, 2007).

Of the VEP components observed following offset of a pattern, P95 amplitude increased with the number of sectors in the pattern. This points to neural response driven by the spatial luminance contrast. Oscillations in the VEP however reveal the presence of high frequencies during the initial part of the VEP, indicating modulation of the neural response by re-entrant projections originating in areas of the dorsal processing stream.

Further, P100 latency did not change with stimulus area undergoing a change in luminance but P1 latency increased. Neither reacted to the number of sectors over which this area was distributed. We will argue that the difference in latency is not an indication for the presence of different mechanism but arises from the ability of the neural response during spatial luminance contrast processing to become manifest in the VEP.

A sustained neural response is reflected poorly in VEP as it is present as a DC shift in the electric potential at the scalp, something the VEP is insensitive to (Marcar et al., 2018; Marcar & Jancke, 2016). Inability by the sustained neural response to become manifest in the VEP leads to it being determined by the phasic neural response only, resulting in a constant P100 latency. The increase in P1 is the result of an interaction between a positive electric dipole generated by temporal luminance contrast processing and a negative electric dipole generated by spatial luminance contrast processing. As the population size involved in temporal luminance contrast processing increases the strength of its electric dipole increases, delaying the moment at which P1 reaches its peak and with it increasing its latency.

Furthermore, N135 and P240 amplitude during spatial luminance contrast processing of a pattern viewed as an alternation display was smaller than N1 and P2 amplitude following onset of the identical windmill (see Graphs 4 & 6). We interpret this as indicating that the neural response processing spatial luminance contrast is a tonic response. A tonic neural response recorded during a pattern alternating display generates a DC shift in the electric potential at the scalp, which manifest poorly in the VEP. N135 and P240 amplitude as well as N1 and P2 amplitude increased with the number of sectors over which the area undergoing a change in luminance but not with size of this area. This links their underlying neural response to spatial luminance contrast processing. That their amplitude no longer reflected the stimulus area undergoing a change in luminance indicates that re-entrant projections altered the spatial distribution of the thalamic activation of striate cortex. This points to areas of the ventral processing stream being the source of the re-entrant projections modulating the neural response in striate cortex during spatial luminance contrast processing.

The view is supported by the prominence of oscillation in the VEP during spatial luminance contrast processing in Θ- and α-range. The presence of slow oscillations indicates modulation of neural activity in striate cortex by re-entrant projections with slow axonal conduction velocity. Longer activation latencies and a coarse or absent retinotopic organisation are features of areas along the ventral processing stream, so that modulation of the neural response in striate cortex during spatial luminance contrast processing is modulated by re-entrant projections originating in areas of the ventral processing stream. The lack of a neural response during spatial luminance contrast processing also accounts for the simpler appearance of the VEP following onset of the disc compared to the other pattern. Other than at its circumference a disc contains no other spatial frequency contrast. N150, P175 and N210 amplitude following offset of a pattern increased as the number of sectors in the display increased. This points to a neural response processing spatial luminance contrast.

Furthermore, N135 latency increased as the size of the stimulus area occupied by white increased but was unaffected by the number of sectors over which this area was distributed. N1 latency decreased as the number of sectors in the pattern increased. As with P100 latency, the reason for this is dipole cancellation. Strengthening of the positive electric dipole during temporal luminance contrast processing shifts the balance between it and the negative electric dipole. As the positive electric dipole gains strength, the time at which the negative electric dipole peaks is moved back in time, resulting in an apparent increase in N135 latency. The decrease in N1 latency as the number of sectors in the pattern increases, is due to a strengthening the negative electric dipole. As the negative electric dipole gains strength, the time at which it peaks is moved forward in time, resulting in a shorter latency.

The decrease in N210 latency as the number of sectors in the pattern increased is an indication for the presence of dipole interaction, with a strengthening of the negative electric dipole moving the time at which it peaks forward in time, generating to a shorter latency.

The example at hand demonstrates that systematic variation of temporal and spatial luminance contrast property of the VEP can provide important insights not only into the mechanism involved in processing the retinal input but also their interaction and the nature of the neural response elicited. Combined with existing knowledge of the anatomical and functional properties of the human visual system the VEP can be used to bridge the gap in understanding on how to link stimulus property and neural activity and provide a means of probing the inner working of the mechanism involved and the way these mechanisms interact to determine the measurable neural response.

In the following further aspects of the present inventions and embodiments thereof are stated as items, wherein the reference numerals in parentheses relate to the Figures. Particularly, these items may also be formulated as claims of the present application.

Item 1: A medical system (20) for measuring visual evoked potentials (VEP) of a person, comprising:
a display (2) for displaying images to be observed by the person with a single eye,
an image generating module (1), the image generating module (1) being configured to cause the display (2) to display a sequence of images comprising a first image, wherein the first image comprises a windmill pattern (30) consisting of a number N of equidistantly spaced light circular sectors (31), and a number of N equidistantly spaced dark circular sectors (32), wherein the number N is selected from the set comprised of 4, 8, 16, 32, and wherein the light and the dark circular sectors (31,32) each extend outwards in a radial direction from a common central point (33), wherein each dark circular sector (32) is arranged between two light circular sectors (31),
a plurality of electrodes (3) configured to be attached to the scalp of the person and to detect EEG signals indicative of electrical activity in the brain of the person in response to observing said sequence of images displayed by the display (2),
an EEG recording module (4) connected to said plurality of electrodes (3), the EEG recording module (4) being configured to record said EEG signals, and
an analyzing module (8), the analyzing module (8) being configured to derive from said EEG signals recorded by the EEG recording module (4) at least one visual evoked potential (VEP).

Item 2: The medical system of item 1, characterized in that the light circular sectors (31) are white circular sectors and/or that the dark circular sectors (32) are black circular sectors

Item 3: The medical system of item 1 or 2, characterized in that the light circular sectors (31) comprise a luminance of about 167 cd/m², and/or wherein the dark circular sectors (32) comprise a luminance of about 0.015 cd/m², and/or wherein the Michelson contrast between light and dark circular sectors (31, 32) is at least 40.

Item 4: The medical system according to one of the preceding items, wherein each light circular sector (31) is delimited by two radii (31a) extending from the common central point (33) in the radial direction and an arc (31b) extending in a circumferential direction.

Item 5: The medical system according to item 4, wherein the arcs (31b) of the light circular sectors (31) are of equal length.

Item 6: The medical system according to one of the preceding items, wherein the light and dark circular sectors (31,32) define a circle.

Item 7: The medical system according to item 6, wherein the first image comprises a region (35) adjacent the circle, said region (35) having the same color and luminance as the dark circular sectors (32) so that the dark circular sectors (32) seamlessly blend into said region (35) of the first image.

Item 8: The medical system according to item 6 or 7, wherein the light circular sectors (31) make up a percentage of the area of the circle, wherein this percentage is one of: 12.5%, 25%, 37.5%, 50%, 75%.

Item 9: The medical system according to one of the preceding items, wherein the image generating module (1) is configured to cause the display (2) to display a sequence of images comprising the first image and a second image, the first and the second image being displayed alternately.

Item 10: The medical system according to item 9, wherein each first image and each second image in the sequence is displayed over a constant period of time, starting with an onset of the respective image and ending with an offset of the respective image.

Item 11: The medical system according to item 10, wherein said constant period of time from onset to offset of the respective image is at least 400 ms, preferably 500 ms.

Item 12: The medical system according to one of the items 9 to 11, wherein the second image corresponds to the first image, but with the order of the light circular sectors (31) and the dark circular sectors (32) being reversed.

Item 13: The medical system according to items 10 and 12, wherein the analyzing module (8) is configured to determine a visual evoked potential (VEP) by averaging a pre-defined number of EEG signal segments following onset of the first and/or second images in the sequence.

Item 14: The medical system according to one of the items 9 to 11, wherein the second image corresponds to an all-dark area having the same color and luminance as the dark circular sectors (32).

Item 15: The medical system according to items 10 and 14, wherein the analyzing module (8) is configured to determine a visual evoked potential (VEP) by averaging a pre-defined number of EEG signal segments following onset of the first images in the sequence, and/or wherein the analyzing module (8) is configured to determine a visual evoked potential (VEP) by averaging a pre-defined number of EEG signal segments following offset of the first images in the sequence.

Item 16: The medical system according to item 13 or 15, wherein the analyzing module (8) is configured to compare an amplitude of at least one deflection of the visual evoked potential (VEP) with an amplitude of a corresponding deflection of a reference curve.

Item 17: The medical system according to item 13 or 15, wherein the analyzing module (8) is configured to compare a latency of at least one deflection of the visual evoked potential (VEP) with a latency of a corresponding deflection of a reference curve.

Item 18: The medical system according to one of the items 16 to 17, wherein the analyzing unit (8) is configured to detect that the person has a diseased retina (10) and to provide a corresponding information to an operator of the system, if the amplitude of said at least one deflection of the visual evoked potential deviates from the amplitude of a corresponding deflection of the reference curve by more than a pre-determined amount, and/or if the latency of said at least one deflection of the visual evoked potential deviates from the latency of a corresponding deflection of the reference curve by more than a pre-determined amount.

Item 19: The medical system according to one of the preceding items, wherein the medical system comprises a camera (5) configured for tracking movement of the eye of the person, wherein the image generation module (1) is configured to only display images of said sequence in case the person fixates said common central point (33) with said eye.

Item 20: A method for measuring visual evoked potentials (VEP) of a person, preferably using a system according to one of the preceding items, the method comprising the steps of:
a) Causing a display (2) to display a sequence of images to be observed by the person with a single eye, the sequence of images comprising a first image, wherein the first image comprises a windmill pattern (30) consisting of a number N of equidistantly spaced light circular sectors (31), and a number N of equidistantly spaced dark circular sectors (32), wherein the number N is selected from the set comprised of 4, 8, 16, 32, and wherein the light and the dark circular sectors (31,32) each extend outwards in a radial direction from a common central point (33), wherein each dark circular sector (32) is arranged between two light circular sectors (31)
b) Detecting EEG signals via electrodes (3) attached to the scalp of the person and recording said EEG signals, the EEG signals being indicative of electrical activity in the brain of the person in response to observing said sequence of images, and
c) Deriving at least one visual evoked potential (VEP) of the person from said EEG signals.

Item 21: The method according to item 20, wherein the light circular sectors (31) are white circular sectors and/or wherein the dark circular sectors (32) are black circular sectors.

Item 22: The method according to one of the items 20 to 21, wherein each light circular sector (31) is delimited by two radii (31a) extending from the common central point (33) in the radial direction and an arc (31b) extending in a circumferential direction.

Item 23: The method according to item 22, wherein the arcs (31b) of the light circular sectors (31) are of equal length.

Item 24: The method according to one of the items 20 to 23, wherein the light and dark circular sectors (31,32) define a circle.

Item 25: The method according to item 24, wherein the first image comprises a region (35) adjacent the circle, said region having the same color and luminance as the dark circular sectors (32) so that the dark circular sectors (32) seamlessly blend into said region (35) of the first image.

Item 26: The method according to item 24 or 25, wherein the light circular sectors (31) make up a percentage of the area of the circle, wherein this percentage is one of: 12.5%, 25%, 37.5%, 50%, 75%.

Item 27: The method according to one of the items 20 to 26, wherein said sequence of images further comprises a second image, the first and the second image being displayed alternately.

Item 28: The method according to item 27, wherein each first image and each second image in the sequence is displayed over a constant period of time, starting with an onset of the respective image and ending with an offset of the respective image.

Item 29: The method according to item 28, wherein said constant period of time from onset to offset of the respective image is at least 400 ms, preferably 500 ms.

Item 30: The medical system according to one of the items 27 to 29, wherein the second image corresponds to the first image, but with the order of the light circular sectors (31) and the dark circular sectors (32) being reversed.

Item 31: The method according to items 28 and 30, wherein a visual evoked potential (VEP) is determined by averaging a pre-defined number of EEG signal segments following onset of the first and/or second images in the sequence.

Item 32: The method according to one of the items 27 to 29, wherein the second image corresponds to an all-dark area having the same color and luminance as the dark circular sectors (32).

Item 33: The method according to items 28 and 32, wherein a visual evoked potential (VEP) is determined by averaging a pre-defined number of EEG signal segments following onset of the first images in the sequence, and/or wherein a visual evoked potential (VEP) is determined by averaging a pre-defined number of EEG signal segments following offset of the first images in the sequence.

Item 34: The method according to item 31 or 33, wherein an amplitude of at least one deflection of the visual evoked potential (VEP) is compared with an amplitude of a corresponding deflection of a reference curve.

Item 35: The method according to item 31 or 33, wherein a latency of at least one deflection of the visual evoked potential (VEP) is compared with a latency of a corresponding deflection of a reference curve.

Item 36: The method according to one of the items 20 to 35, wherein a movement of the eye of the person is tracked, wherein images of said sequence are only displayed in case the person fixates said common central point (33) with said eye.

Item 37: A computer program, the computer program comprising instructions which, when the program is executed by a computer, cause the computer to carry out step a) of the method according to one of the items 20 to 36.

1. A medical system (20) for measuring visual evoked potentials (VEP) of a person, comprising:
a display (2) for displaying images to be observed by the person with a single eye,
an image generating module (1), the image generating module (1) being configured to cause the display (2) to display a sequence of images comprising a first image and a second image, the first and the second image being displayed alternately, wherein the first image comprises a windmill pattern (30) consisting of a number N of equidistantly spaced light circular sectors (31), and a number of N equidistantly spaced dark circular sectors (32), wherein the number N is selected from the set comprised of 4, 8, 16, 32, and wherein the light and the dark circular sectors (31,32) each extend outwards in a radial direction from a common central point (33), wherein each dark circular sector (32) is arranged between two light circular sectors (31), wherein the second image corresponds to the first image, but with the order of the light circular sectors (31) and the dark circular sectors (32) being reversed, or wherein the second image corresponds to an all-dark area having the same color and luminance as the dark circular sectors (32),
a plurality of electrodes (3) configured to be attached to the scalp of the person and to detect EEG signals indicative of electrical activity in the brain of the person in response to observing said sequence of images displayed by the display (2),
an EEG recording module (4) connected to said plurality of electrodes (3), the EEG recording module (4) being configured to record said EEG signals, and an analyzing module (8), the analyzing module (8) being configured to derive from said EEG signals recorded by the EEG recording module (4) at least one visual evoked potential (VEP).

2. The medical system of claim 1, characterized in that the light circular sectors (31) are white circular sectors and/or that the dark circular sectors (32) are black circular sectors.

3. The medical system of claim 1, characterized in that the light circular sectors (31) comprise a luminance of about 167 cd/m2, and/or wherein the dark circular sectors (32) comprise a luminance of about 0.015 cd/m2, and/or wherein the Michelson contrast between light and dark circular sectors (31, 32) is at least 40.

4. The medical system according to claim 1, wherein each light circular sector (31) is delimited by two radii (31a) extending from the common central point (33) in the radial direction and an arc (31b) extending in a circumferential direction.

5. The medical system according to claim 4, wherein the arcs (31b) of the light circular sectors (31) are of equal length.

6. The medical system according to claim 1, wherein the light and dark circular sectors (31,32) define a circle.

7. The medical system according to claim 6, wherein the first image comprises a region (35) adjacent the circle, said region (35) having the same color and luminance as the dark circular sectors (32) so that the dark circular sectors (32) seamlessly blend into said region (35) of the first image.

8. The medical system according to claim 6, wherein the light circular sectors (31) make up a percentage of the area of the circle, wherein preferably this percentage is one of: 12.5%, 25%, 37.5%, 50%, 75%.

9. The medical system (20) according to claim 8, wherein the image generating module (1) is configured to cause the display (2) to display a series of sequences for different percentages of the area of the circle.

10. The medical system according to claim 9, wherein each first image and each second image in the sequence is displayed over a constant period of time, starting with an onset of the respective image and ending with an offset of the respective image.

11. The medical system according to claim 10, wherein said constant period of time from onset to offset of the respective image is at least 400 ms, preferably 500 ms.

12. The medical system according to claim 10, wherein the analyzing module (8) is configured to determine a visual evoked potential (VEP) by averaging a pre-defined number of EEG signal segments following onset of the first and/or second images in the sequence.

13. The medical system according to claim 1, wherein the analyzing module (8) is configured to determine a visual evoked potential (VEP) by averaging a pre-defined number of EEG signal segments following onset of the first images in the sequence, and/or wherein the analyzing module (8) is configured to determine a visual evoked potential (VEP) by averaging a pre-defined number of EEG signal segments following offset of the first images in the sequence.

14. The medical system according to claim 12, wherein the analyzing module (8) is configured to compare an amplitude of at least one deflection of the visual evoked potential (VEP) with an amplitude of a corresponding deflection of a reference curve.

15. The medical system according to claim 12, wherein the analyzing module (8) is configured to compare a latency of at least one deflection of the visual evoked potential (VEP) with a latency of a corresponding deflection of a reference curve.

16. The medical system according to claim 14, wherein the analyzing unit (8) is configured to detect that the person has a diseased retina (10) and to provide a corresponding information to an operator of the system, if the amplitude of said at least one deflection of the visual evoked potential deviates from the amplitude of a corresponding deflection of the reference curve by more than a pre-determined amount, and/or if the latency of said at least one deflection of the visual evoked potential deviates from the latency of a corresponding deflection of the reference curve by more than a pre-determined amount.

17. The medical system according to claim 1, wherein the medical system comprises a camera (5) configured for tracking movement of the eye of the person, wherein the image generation module (1) is configured to only display images of said sequence in case the person fixates said common central point (33) with said eye.

18. The medical system (20) according to claim 16, wherein the analyzing unit (8) is configured to detect that the person has a diseased retina (10) based on comparing amplitudes of a P100 deflection of the visual evoked potential (VEP) obtained from displaying a series of sequences for different percentages of the area of the circle to be observed by the person with a reference curve.

19. The medical system (20) according to claim 18, wherein the reference curve is a linear function of the percentage of the area of the circle.

20. A method for measuring visual evoked potentials (VEP) of a person, preferably using a system according to claim 1, the method comprising the steps of:
a) Causing a display (2) to display a sequence of images to be observed by the person with a single eye, the sequence of images comprising a first image and a second image, the first and the second image being displayed alternately, wherein the first image comprises a windmill pattern (30) consisting of a number N of equidistantly spaced light circular sectors (31), and a number N of equidistantly spaced dark circular sectors (32), wherein the number N is selected from the set comprised of 4, 8, 16, 32, and wherein the light and the dark circular sectors (31,32) each extend outwards in a radial direction from a common central point (33), wherein each dark circular sector (32) is arranged between two light circular sectors (31), wherein the second image corresponds to the first image, but with the order of the light circular sectors (31) and the dark circular sectors (32) being reversed, or wherein the second image corresponds to an all-dark area having the same color and luminance as the dark circular sectors (32),
b) Detecting EEG signals via electrodes (3) attached to the scalp of the person and recording said EEG signals, the EEG signals being indicative of electrical activity in the brain of the person in response to observing said sequence of images, and
c) Deriving at least one visual evoked potential (VEP) of the person from said EEG signals.

21. The method according to claim 20, wherein the light circular sectors (31) are white circular sectors and/or wherein the dark circular sectors (32) are black circular sectors.

22. The method according to claim 20, wherein each light circular sector (31) is delimited by two radii (31a) extending from the common central point (33) in the radial direction and an arc (31b) extending in a circumferential direction.

23. The method according to claim 22, wherein the arcs (31b) of the light circular sectors (31) are of equal length.

24. The method according to claim 20, wherein the light and dark circular sectors (31,32) define a circle.

25. The method according to claim 24, wherein the first image comprises a region (35) adjacent the circle, said region having the same color and luminance as the dark circular sectors (32) so that the dark circular sectors (32) seamlessly blend into said region (35) of the first image.

26. The method according to claim 24, wherein the light circular sectors (31) make up a percentage of the area of the circle, wherein this percentage is one of: 12.5%, 25%, 37.5%, 50%, 75%.

27. The method according to claim 20, wherein each first image and each second image in the sequence is displayed over a constant period of time, starting with an onset of the respective image and ending with an offset of the respective image.

28. The method according to claim 27, wherein said constant period of time from onset to offset of the respective image is at least 400 ms, preferably 500 ms.

29. The method according to claim 27, wherein a visual evoked potential (VEP) is determined by averaging a pre-defined number of EEG signal segments following onset of the first and/or second images in the sequence.

30. The method according to claim 27, wherein a visual evoked potential (VEP) is determined by averaging a pre-defined number of EEG signal segments following onset of the first images in the sequence, and/or wherein a visual evoked potential (VEP) is determined by averaging a pre-defined number of EEG signal segments following offset of the first images in the sequence.

31. The method according to claim 29, wherein an amplitude of at least one deflection of the visual evoked potential (VEP) is compared with an amplitude of a corresponding deflection of a reference curve.

32. The method according to claim 29, wherein a latency of at least one deflection of the visual evoked potential (VEP) is compared with a latency of a corresponding deflection of a reference curve.

33. The method according to claim 20, wherein a movement of the eye of the person is tracked, wherein images of said sequence are only displayed in case the person fixates said common central point (33) with said eye.

34. The method according to claim 20, wherein amplitudes of a P100 deflection of the visual evoked potential (VEP) obtained from displaying a series of sequences for different percentages of the area of the circle are compared to a reference curve.

35. The method according to claim 34, wherein the reference curve is a linear function of the percentage of the area of the circle.

36. A computer program, the computer program comprising instructions which, when the program is executed by a computer, cause the computer to carry out step a) of the method according to claim 20.
